(19) United States
(12) Patent Application Publication
(10) Pub. No.: US 2015/0216445 A1
(43) Pub. Date: Aug. 6, 2015

(54) SYSTEM AND METHOD FOR ACCURATE PLACEMENT OF A CATHETER TIP IN A PATIENT

(71) Applicant: ELCAM MEDICAL AGRICULTURAL COOPERATIVE ASSOCIATION LTD., Merom Hagalil (IL)

(72) Inventors: Moni Carmeli, Kiriat Yam (IL); Ran Ginsburg, Ramat Gan (IL)

(73) Assignee: ELCAM MEDICAL AGRICULTURAL COOPERATIVE ASSOCIATION LTD., Merom Hagalil (IL)

(21) Appl. No.: 14/409,879

(22) PCT Filed: Jul. 28, 2013
(86) PCT No.: PCT/IL13/50640

§ 371 Date: Dec. 19, 2014

Related U.S. Application Data
(60) Provisional application No. 61/683,216, filed on Aug. 15, 2012.

(51) Int. Cl. A61B 5/06; A61B 5/0402; A61B 5/00; A61B 5/0215

(52) U.S. Cl. CPC A61B 5/061; A61B 5/0215; A61B 5/0402; A61B 5/7246; A61B 5/742; A61B 5/7405

(57) ABSTRACT

A system for providing indications of location of a catheter tip within a patient, the system including a memory storing synthesized blood pressure waveform parameters representative of a first plurality of catheter tip locations in a second plurality of representative patients, real time patient blood pressure waveform parameter acquisition circuitry operative to acquire real time blood pressure waveform parameters of a patient currently undergoing catheterization, which parameters contain information useful in ascertaining the location of a catheter tip in the patient and real time patient catheter tip location ascertaining circuitry operative to receive the real time patient blood pressure waveform parameters from the acquisition circuitry and to employ the synthesized blood pressure waveform parameters for providing a real time indication of catheter tip location in the patient.

### REFERENCE TO RELATED APPLICATIONS

Reference is hereby made to U.S. Provisional Patent Application Ser. No. 61/683,216, filed Aug. 15, 2012, entitled SYSTEM AND METHOD FOR ACCURATE PLACEMENT OF A CATHETER TIP IN A PATIENT, the disclosure of which is hereby incorporated by reference and priority of which is hereby claimed pursuant to 37 CFR 1.78(a) (4) and (5)(i).

Reference is also made to U.S. patent application Ser. No. 12/594,869, filed Apr. 9, 2008 and entitled “SYSTEM AND METHOD FOR ACCURATE PLACEMENT OF A CATHETER TIP IN A PATIENT”, the disclosure of which is hereby incorporated by reference.

### FIELD OF THE INVENTION

The present invention relates to medical devices and methodologies generally and more particularly to devices and methodologies for accurate catheter tip placement in a patient.

### BACKGROUND OF THE INVENTION

Accurate placement of a catheter tip in a patient is an important factor in the effective treatment of many conditions.

### SUMMARY OF THE INVENTION

The present invention seeks to provide an improved system and methodology for accurate placement of a catheter tip inside a patient.

There is thus provided in accordance with a preferred embodiment of the present invention a system for providing indications of location of a catheter tip within a patient, the system including a memory storing synthesized blood pressure waveform parameters representative of a first plurality of catheter tip locations in a second plurality of representative patients, real time patient blood pressure waveform parameter acquisition circuitry operative to acquire real time blood pressure waveform parameters of a patient currently undergoing catheterization, which parameters contain information useful in ascertaining the location of a catheter tip in the patient and real time patient catheter tip location ascertaining circuitry operative to receive the real time patient blood pressure waveform parameters from the acquisition circuitry and to employ the synthesized blood pressure waveform parameters for providing a real time indication of catheter tip location in the patient.

Preferably, the synthesized blood pressure waveform parameters are based on waveform parameters from multiple ones of the plurality of representative patients.

In accordance with a preferred embodiment of the present invention, the real time patient blood pressure waveform parameter acquisition circuitry is operative to acquire real time blood pressure waveform parameters of the plurality of representative patients and the system also includes patient blood pressure waveform parameter grouping circuitry operative to group the plurality of representative patients into multiple groups and blood pressure waveform parameter synthesizing circuitry operative to generate the synthesized blood pressure waveform parameters for each of the multiple groups based on the blood pressure waveform parameters of the plurality of representative patients.

Preferably, the synthesized blood pressure waveform parameters are grouped into multiple groups having mutually differing relationships between measured and estimated catheter tip locations and the real time patient catheter tip location ascertaining circuitry is operative to associate parameters of a patient with parameters of one of the multiple groups in order to enhance accuracy of indication of catheter tip location based on the real time patient blood pressure waveform parameters.

In accordance with a preferred embodiment of the present invention, the multiple groups differ in characteristic blood pressure signal propagation rate within a vein of the patient.

Preferably, the real time patient catheter tip location ascertaining circuitry is operative to match the real time patient blood pressure waveform parameters with at least one of the synthesized blood pressure waveform parameters for providing a real time indication of catheter tip location in the patient. Additionally, the real time patient catheter tip location ascertaining circuitry employs cross-correlation analysis.

In accordance with a preferred embodiment of the present invention, the system also includes electrodes located on the patient skin, the electrodes being operative to provide ECG waveform parameters of the patient.

Preferably, the real time blood pressure waveform parameters are recorded in synchronization with the ECG waveform parameters and an R wave peak of the ECG waveform parameters represents a fiducial point in time with respect to which the real time blood pressure waveform parameters are measured. Additionally, the time shift between the real time blood pressure waveform parameters and the R wave peak of the ECG waveform parameters provides a real time indication of catheter tip location in the patient.

In accordance with a preferred embodiment of the present invention, the real time patient blood pressure waveform parameter acquisition circuitry includes a pressure transducer operatively coupled to the catheter tip.

Preferably, the real time patient catheter tip location ascertaining circuitry is also operative to provide at least one catheter tip insertion instruction to an operator based on the real time indication of catheter tip location in the patient. Additionally, the system also includes at least one of a display and an audio output device and the at least one catheter tip insertion instruction is provided to the operator via the at least one of a display and an audio output device.

There is also provided in accordance with another preferred embodiment of the present invention a method for providing indications of location of a catheter tip within a patient, the method including storing synthesized blood pressure waveform parameters representative of a first plurality of catheter tip locations in a second plurality of representative patients, acquiring real time patient blood pressure waveform parameters of a patient currently undergoing catheterization, which parameters contain information useful in ascertaining the location of a catheter tip in the patient and ascertaining a real time indication of catheter tip location in the patient by employing the real time patient blood pressure waveform parameters and the synthesized blood pressure waveform parameters.

Preferably, the method also includes automatically generating the synthesized blood pressure waveform parameters based on waveform parameters from multiple ones of the plurality of representative patients. Additionally, the automatically generating includes acquiring real time patient blood pressure waveform parameters of the plurality of representative patients, grouping the plurality of representative patients into multiple groups and generating the synthesized blood pressure waveform parameters based on the blood pressure waveform parameters for each of the multiple groups.

In accordance with a preferred embodiment of the present invention, the multiple groups have mutually differing relationships between measured and estimated catheter tip locations and the ascertaining includes associating blood pressure waveform parameters of a patient with blood pressure waveform parameters of one of the multiple groups in order to enhance accuracy of indication of catheter tip location. Additionally or alternatively, the ascertaining includes matching the real time patient blood pressure waveform parameters with at least one of the synthesized blood pressure waveforms parameters.

Preferably, the multiple groups differ in characteristic blood pressure signal propagation rate within the vein of the patient.

In accordance with a preferred embodiment of the present invention, the method also includes acquiring real time patient ECG waveform parameters of the patient.

Preferably, the acquiring real time patient blood pressure waveform parameters of a patient and the acquiring real time patient ECG waveform parameters of the patient includes synchronized acquiring and an R wave peak of the ECG waveform parameters represents a fiducial point in time with respect to which the real time blood pressure waveform parameters are measured.

In accordance with a preferred embodiment of the present invention, the ascertaining includes ascertaining a time shift between the real time blood pressure waveform parameters and the R wave peak of the ECG waveform parameters. Additionally or alternatively, the ascertaining a real time indication of catheter tip location in the patient includes employing a cross-correlation analysis.

Preferably, the ascertaining real time patient catheter tip location also includes providing at least one catheter tip insertion instruction to an operator based on the real time indication of catheter tip location in the patient. Additionally, the at least one catheter tip insertion instruction is provided to the operator via at least one of a display and an audio output device.

### BRIEF DESCRIPTION OF THE DRAWINGS

The present invention will be understood and appreciated more fully from the following detailed description, taken in conjunction with the drawings in which:

FIGS. 1A-1F are together a simplified illustrated flowchart illustrating a methodology for obtaining representative blood pressure waveforms, having an ECG-based fiducial, associated with insertion of a catheter at the right atrium/superior vena cava junction in accordance with a preferred embodiment of the present invention;

FIG. 2 is a simplified illustration of a pre-clinical procedure for obtaining representative blood pressure waveforms, having an ECG-based fiducial;

FIG. 3 is a simplified illustration of a clinical procedure for precise insertion of a catheter at the right atrium/superior vena cava junction;

FIGS. 4A-4C are together a simplified illustrated flowchart illustrating a methodology for precise insertion of a catheter at the right atrium/superior vena cava junction in accordance with a preferred embodiment of the present invention;

FIG. 5 is a simplified block diagram illustration of the system of a preferred embodiment of the present invention.

### DETAILED DESCRIPTION OF A PREFERRED EMBODIMENT

The present invention relates to a system and methodology for precise insertion of a catheter at the right atrium/superior vena cava junction of a patient.

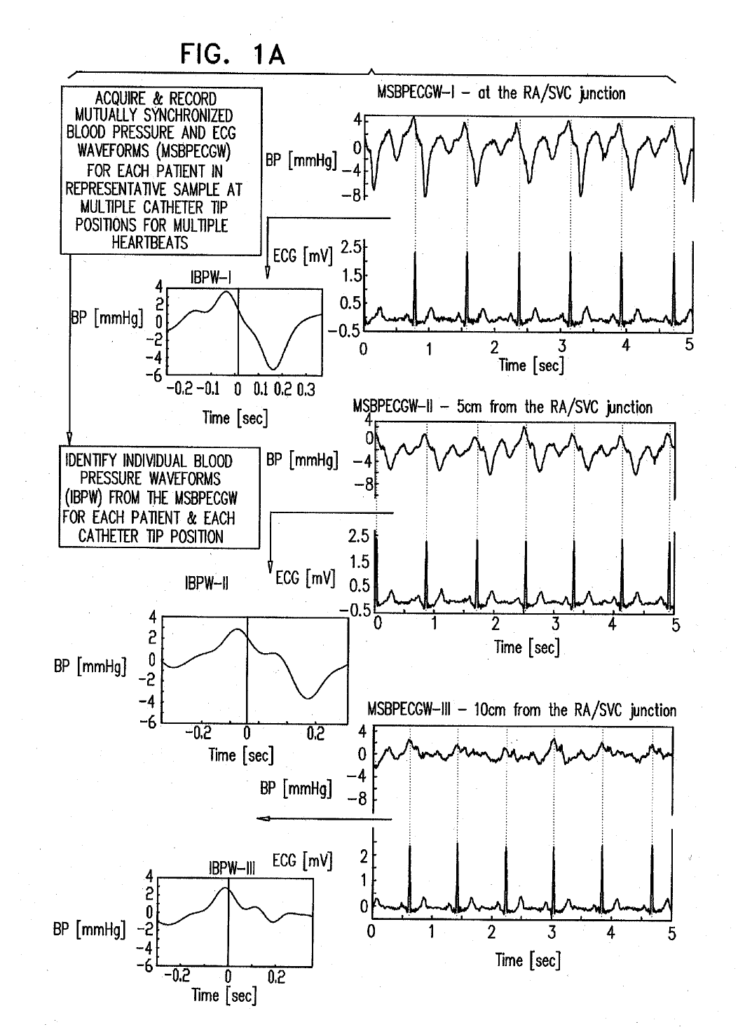

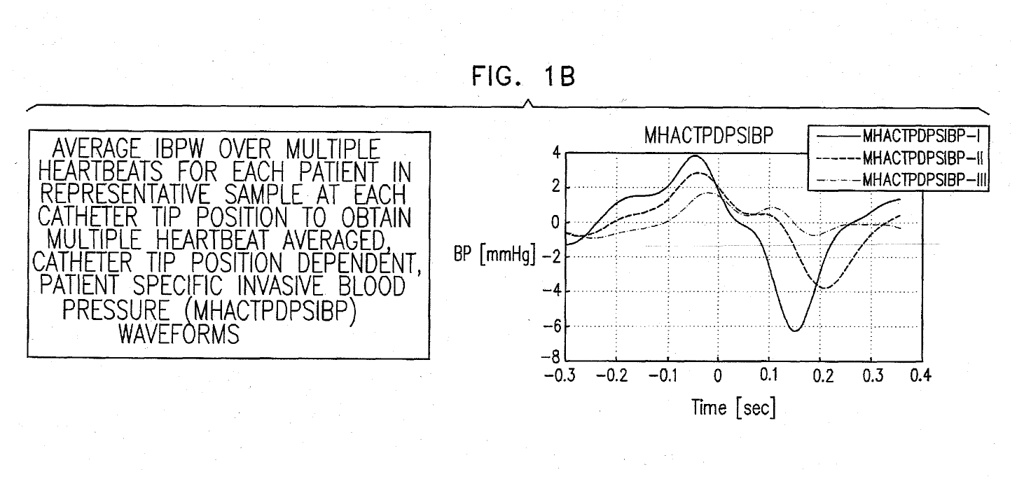

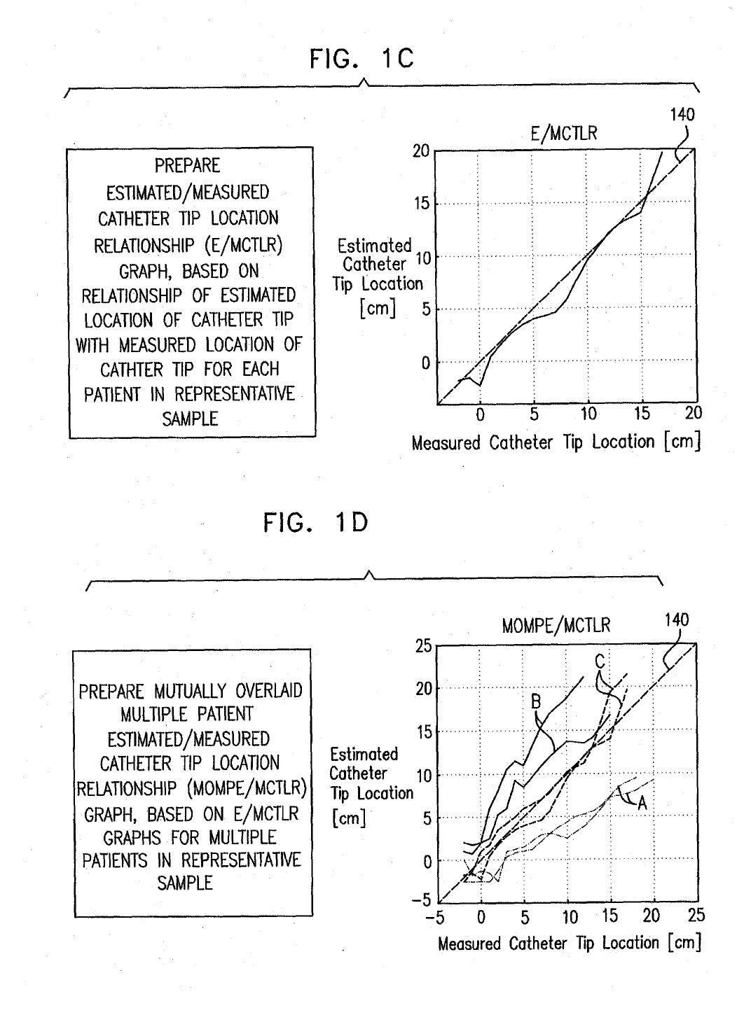

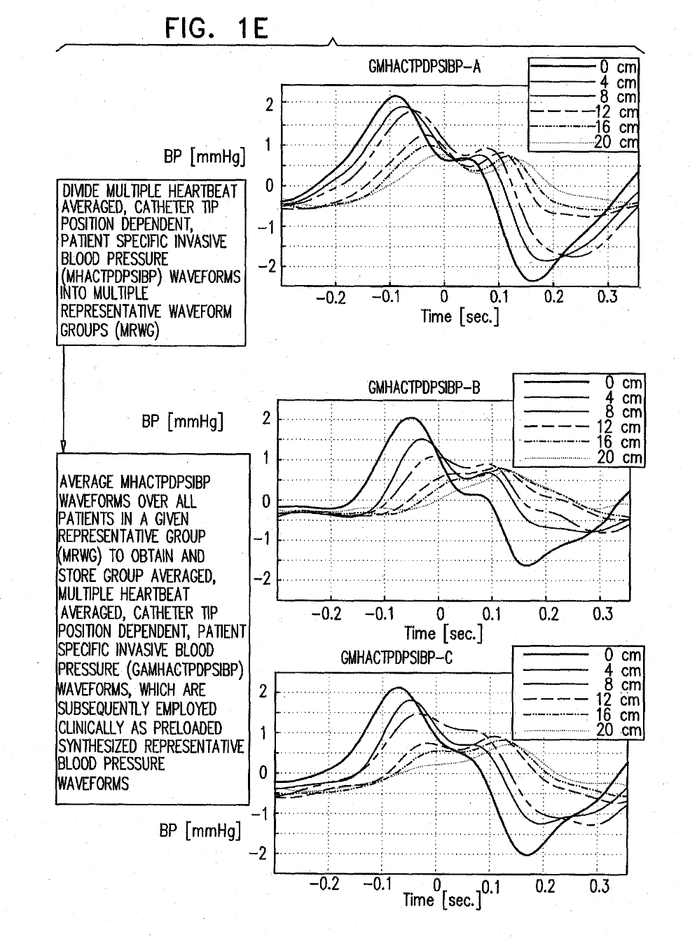

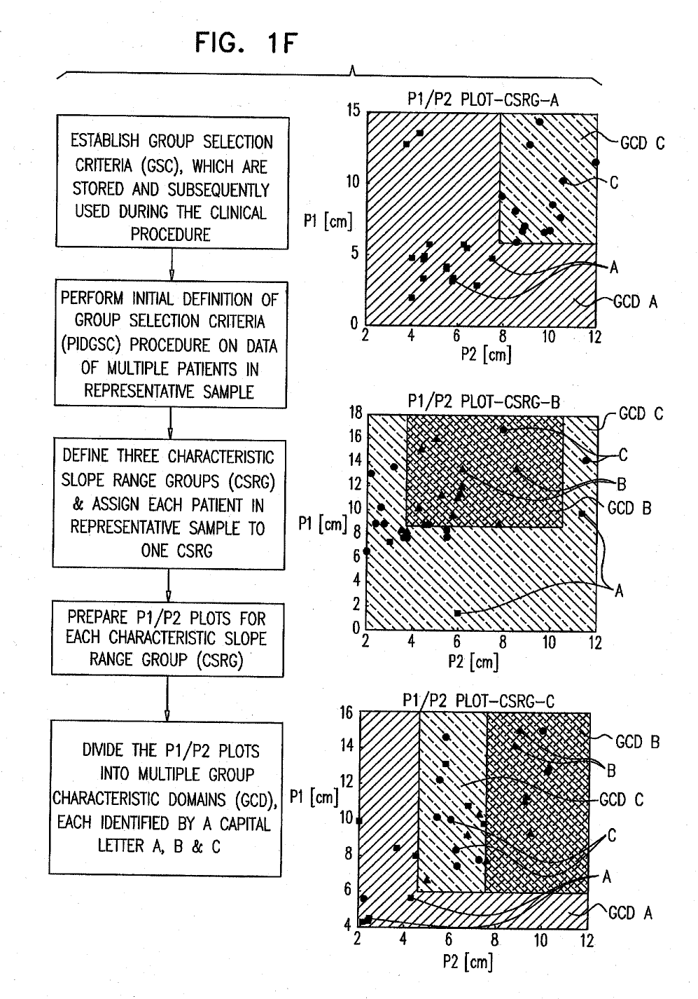

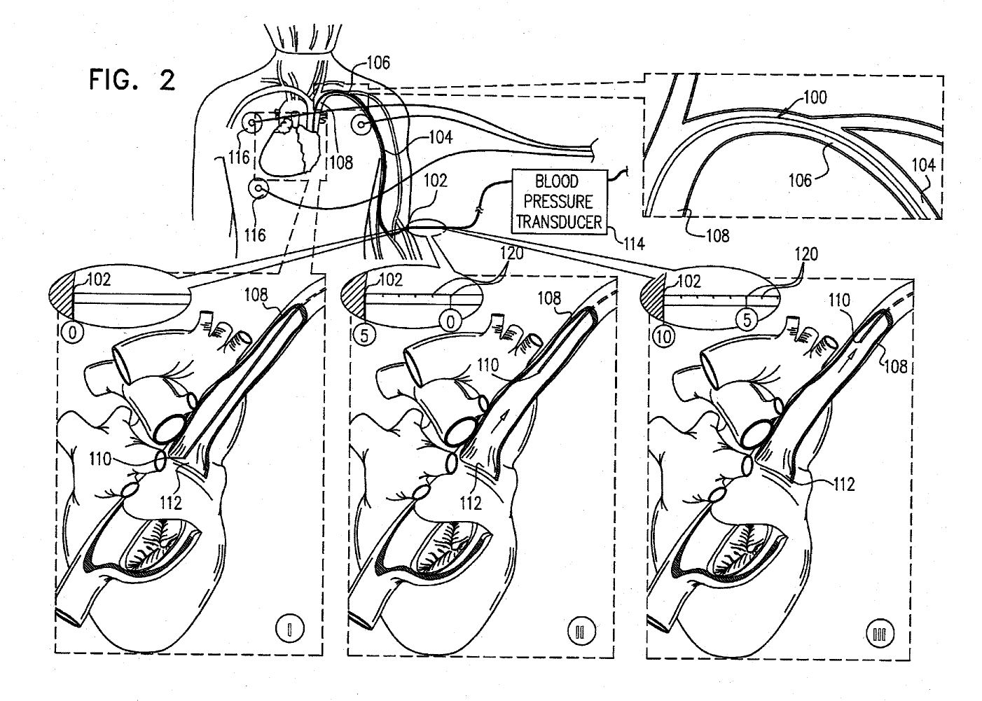

Reference is now made to FIGS. 1A-1F, which are together a simplified illustrated flowchart illustrating a methodology for obtaining representative blood pressure waveforms, having an ECG-based fiducial, associated with insertion of a catheter at the right atrium/superior vena cava junction in accordance with a preferred embodiment of the present invention, and to FIG. 2, which is a simplified illustration of a pre-clinical procedure for obtaining representative blood pressure waveforms, having an ECG-based fiducial, which preferably is used in the methodology of FIGS. 1A-1F.

The methodology of the present invention enables precise insertion of a catheter at the right atrium/superior vena cava junction by employing preloaded synthesized representative blood pressure waveforms corresponding to a plurality of catheter tip locations relative to the right atrium/superior vena cava junction and matching a current sensed blood pressure waveform with one or more of the preloaded synthesized representative blood pressure waveforms, thereby to provide an indication of current catheter tip location. Based on the indication of current catheter tip location, one or more instructions to an operator may be provided, directing the operator to displace the catheter until a desired catheter tip location is reached.

Reference is now made specifically to FIGS. 1A-1F, which illustrate an initial series of steps in a methodology for obtaining representative blood pressure waveforms, having an ECG-based fiducial. The methodology of FIGS. 1A-1F is carried out at a pre-clinical stage at which synthesis of the synthesized representative blood pressure waveforms takes place. Generally speaking, the synthesis is based on clinical experience with a representative sample of patients having a range of different personal parameters, such as age, sex, height, weight and race. Typically, the patients, whose data is employed in the synthesis, are undergoing peripheral inserted central catheterization (PICC) for the purpose of administration of drugs at a relatively precise location relative to the heart.

As seen in FIG. 2, in the course of the PICC procedure, a catheter 100 is inserted, typically at an insertion location 102 at the patient's arm and through a vein in the arm to the axillary vein 104, thence to the subclavian vein 106 into the superior vena cava 108 until the tip 110 of the catheter 100 reaches the right atrium/superior vena cava junction 112. The actual position of the tip 110 at the right atrium/superior vena cava junction 112 is preferably verified by X-ray imaging. During this procedure the parameters of the patient's heartbeat in the form of an ECG signal are received and recorded.

Beginning when the catheter tip 110 is at the right atrium/superior vena cava junction 112, as seen at enlargement I in FIG. 2, and thereafter, as the catheter tip 110 is stepwise withdrawn towards the axillary vein 104, the blood pressure waveform and the ECG waveform of the patient are recorded in mutual synchronization, where preferably the R wave peak of the ECG waveform represents a fiducial point in time with respect to which the blood pressure waveform is measured. Typically, the blood pressure waveform at the catheter tip 110 is acquired by a blood pressure transducer 114 at a proximal end of the catheter 100, the blood pressure waves passing through liquid in the catheter 100 from the tip 110 to the transducer 114. Typically, the ECG waveform is acquired by several surface electrodes 116 (FIG. 5) that are positioned on the patient's skin, for example on the patient's chest, as seen in FIG. 2, or, alternatively, on the patient's right arm, left arm and left leg.

An example of mutually synchronized blood pressure and ECG waveforms (MSBPECGW) for a typical patient, recorded when tip 110 of the catheter 100 is located at the right atrium/superior vena cava junction 112, appears at MSBPECGW-I in FIG. 1A. Typically, mutually synchronized blood pressure and ECG waveforms are recorded for a plurality of heartbeats, typically at least 20 heartbeats, at each of the stepwise positions of the catheter tip 110 as it is withdrawn from the right atrium/superior vena cava junction 112 towards the axillary vein 104.

As seen in FIG. 2, the catheter 100 typically has markings, generally designated by reference numeral 120, at 1 cm intervals therealong, which markings can be readily seen by the operator. As seen at enlargement I in FIG. 2, typically when the catheter tip is at the right atrium/superior vena cava junction 112, the catheter marking 0 cm appears at the insertion location 102. The operator withdraws the catheter typically in steps, each of between 1 cm-2.5 cm, preferably each of 1 cm, and the mutually synchronized blood pressure and ECG waveforms are recorded for a plurality of heartbeats, typically at least 20 heartbeats, at the position of the catheter tip 110 at each step, as indicated by markings 120.

Two non-sequential typical positions of the catheter tip 110 are illustrated in FIG. 2, at enlargements II and III respectively, and typical mutually synchronized blood pressure and ECG waveforms (MSBPECGW) for these two positions appear at MSBPECGW-II and MSBPECGW-III in FIG. 1A. It is noted from a visual comparison of the mutually synchronized blood pressure and ECG waveforms appearing as MSBPECGW-I, MSBPECGW-II and MSBPECGW-III in FIG. 1A, that there is a significant time shift in the time relationship between the blood pressure waveform and the heart beat timing, represented by the R wave peak of the ECG signal, which time shift represents the distance of the catheter tip 110 from the right atrium/superior vena cava junction 112. As seen in the illustrated embodiment at enlargements II and III in FIG. 2, the catheter tip has been moved 5 cm and 10 cm, respectively, from the right atrium/superior vena cava junction 112, and the catheter marking reading 0 cm has correspondingly moved 5 cm and 10 cm, respectively.

The mutually synchronized blood pressure and ECG waveforms (MSBPECGW) taken from the representative sample of patients are employed for two distinct purposes:

Provision of Group Averaged, Multiple Heartbeat Averaged Catheter Tip Position Dependent, Patient Specific Invasive Blood Pressure (GAMHACTPDPSIBP) waveforms, which are stored and later used in a clinical procedure; and

Establishment of Group Selection Criteria (GSC), which are stored and later used during the clinical procedure.

Provision of Group Averaged, Multiple Heartbeat Averaged Catheter Tip Position Dependent, Patient Specific Invasive Blood Pressure (GAMHACTPDPSIBP) waveforms preferably takes place as follows:

For each mutually synchronized blood pressure and ECG waveforms (MSBPECGW) recorded for each patient at each catheter tip position, a plurality of individual blood pressure waveforms (IBPW) is preferably generated by dividing the blood pressure waveform recorded for the plurality of heartbeats into individual blood pressure waveforms of single heartbeat length, using the R wave peak of the ECG waveform as a fiducial point in time with respect to the blood pressure waveform. The individual blood pressure waveforms (IBPW) are bounded in time by a fixed time window, typically of duration approximately 660 ms, which preferably opens 300 ms prior to each detected R wave peak in the Mutually Synchronized Blood Pressure and ECG Waveforms (MSBPECGW). Additionally, the individual blood pressure waveforms (IBPW) are preferably filtered using conventional filtering techniques, such as Finite Impulse Response (FIR). Typical individual blood pressure waveforms (IBPW), corresponding to MSBPECGW-I, MSBPECGW-II and MSBPECGW-III, are shown as IBPW-I, IBPW-II and IBPW-III in FIG. 1A.

As seen in FIG. 1B, preferably, for each patient in the representative sample and for each position at which the mutually synchronized blood pressure and ECG waveforms (MSBPECGW) are recorded for multiple heartbeats, a Multiple Heartbeat Averaged, Catheter Tip Position Dependent, Patient Specific Invasive Blood Pressure (MHACTPDPSIBP) waveform is calculated, by averaging at least two, and preferably all, of the plurality of individual blood pressure waveforms (IBPW), and stored. Typical MHACTPDPSIBP waveforms, corresponding to the positions shown at I, II and III in FIG. 2, appear in FIG. 1B and are designated MHACTPDPSIBP-I, MHACTPDPSIBP-II and MHACTPDPSIBP-III, respectively.

Preferably, a linear interpolation function is subsequently applied to the calculated MHACTPDPSIBP waveforms for each patient to provide additional interpolated waveforms representative of waveforms at additional catheter tip locations. Including the additional interpolated waveforms, at least 20 MHACTPDPSIBP waveforms per patient, corresponding to at least 20 different catheter tip positions, are calculated.

The calculated MHACTPDPSIBP and the interpolated MHACTPDPSIBP waveforms taken from the representative sample of patients are divided into multiple representative waveform groups (MRWG) according to a characteristic blood pressure signal propagation rate within the patient's vein. It is appreciated that the calculated blood pressure waveforms of groups with different characteristic blood pressure signal propagation rates will typically have different shapes.

Definition of the multiple representative waveform groups (MRWG) preferably is carried out as follows:

First, all of the patients in the representative sample are grouped together. Preferably, each of the at least 20 MHACTPDPSIBP waveforms per patient, which correspond to at least 20 different positions of the catheter tip 110, is averaged over all of the patients in the sample, thereby producing at least 20 Sample Averaged, Multiple Heartbeat Averaged Catheter Tip Position Dependent, Patient Specific Invasive Blood Pressure (SAMHACTPDPSIBP) waveforms, which correspond to at least 20 different positions of the catheter tip 110 in an average patient in the sample. The at least 20 Sample Averaged, Multiple Heartbeat Averaged Catheter Tip Position Dependent, Patient Specific Invasive Blood Pressure (SAMHACTPDPSIBP) waveforms are stored and employed in the subsequent steps.

For each patient in the representative sample, an

Estimated/Measured Catheter Tip Location Relationship (E/MCTLR) graph is then prepared, which relates an estimated location of the catheter tip 110 with the measured location of the catheter tip 110 based on the initial X-ray verified position of the catheter tip 110 and the corresponding and subsequent readings of markings 120 on the catheter 100. An example of an Estimated/Measured Catheter Tip Location Relationship graph for a given patient, including an identity line 140, indicating points at which the estimated location of the catheter tip 110 is identical to the measured location of the catheter tip 110, appears in FIG. 1C and is designated E/MCTLR.

The estimated locations of the catheter tip 110 which are used in the E/MCTLR graph are obtained in a manner analogous to the method described hereinbelow for calculating Median Simulated Estimated Catheter Tip Location (MSECTL), based on a simulated catheter insertion procedure based on the individual blood pressure waveforms (IBPW) of the given patient, except that the estimated locations used in the E/MCTLR graph are calculated using the Sample Averaged, Multiple Heartbeat Averaged Catheter Tip Position Dependent, Patient Specific Invasive Blood Pressure (SAMHACTPDPSIBP) waveforms instead of the Group Averaged, Multiple Heartbeat Averaged Catheter Tip Position Dependent, Patient Specific Invasive Blood Pressure (GAMHACTPDPSIBP) waveforms used below in calculating the MSECTLs.

The measured location of the catheter tip 110 used is based on the initial X-ray verified position of the catheter tip 110 and the subsequent readings of markings 120 of the catheter 100 at which the stored tip position location specific corresponding plurality of individual blood pressure waveforms (IBPW) were obtained.

An E/MCTLR graph is preferably prepared for each of the patients in the representative sample and the multiple E/MCTLR graphs of the multiple patients in the representative sample are mutually overlaid onto a single graph. An example of mutually overlaid multiple E/MCTLR graphs, including an identity line 140, indicating points at which the estimated location of the catheter tip 110 is identical to the measured location of the catheter tip 110, appears in FIG. 1D, and is designated as a Mutually Overlaid Multiple Patient Estimated/Measured Catheter Tip Location Relationship (MOMPE/MCTLR) graph.

From a consideration of the Mutually Overlaid Multiple Patient Estimated/Measured Catheter Tip Location Relationship (MOMPE/MCTLR) graph, it is seen that for a first group of patients, designated A, the ratio of Estimated Catheter Tip Location to the Measured Catheter Tip Location generally consistently appears below the identity line 140 and for a second group of patients, designated B, the ratio of Estimated Catheter Tip Location to the Measured Catheter Tip Location generally consistently appears above the identity line 140. For a third group of patients, designated C, the ratio of Estimated Catheter Tip Location to the Measured Catheter Tip Location is sometimes above and sometimes below the identity line 140. The three groups of patients thus defined are preferably employed as the multiple representative waveform groups (MRWG). It is appreciated that other definitions and methodologies may alternatively be used to define the multiple representative waveform groups (MRWG).

For example, if approximately 100 patients make up the representative sample, three groups may be appropriate. Preferably, each of the at least 20 MHACTPDPSIBP waveforms per patient in a given group, which correspond to at least 20 different positions of the catheter tip 110, is averaged over all of the patients in the group, thereby producing at least 20 Group Averaged, Multiple Heartbeat Averaged Catheter Tip Position Dependent, Patient Specific Invasive Blood Pressure (GAMHACTPDPSIBP) waveforms per group, which correspond to at least 20 different positions of the catheter tip 110 in an average patient in the group.

The at least 20 Group Averaged, Multiple Heartbeat Averaged Catheter Tip Position Dependent, Patient Specific Invasive Blood Pressure (GAMHACTPDPSIBP) waveforms are stored and subsequently employed clinically as the aforementioned preloaded synthesized representative blood pressure waveforms. Typical Group Averaged, Multiple Heartbeat Averaged Catheter Tip Position Dependent, Patient Specific Invasive Blood Pressure (GAMHACTPDPSIBP) waveform per catheter tip location corresponding to each of the multiple representative waveform groups (MRWG) appear in FIG. 1E and are designated GAMHACTPDPSIBP-A, GAMHACTPDPSIBP-B and GAMHACTPDPSIBP-C, respectively.

Establishment of Group Selection Criteria (GSC) preferably is carried out based on the Group Averaged, Multiple Heartbeat Averaged Catheter Tip Position Dependent, Patient Specific Invasive Blood Pressure (GAMHACTPDPSIBP) waveforms, provided as described hereinabove, by performing a data simulation of the clinical procedure for each of the patients in the representative sample as described in detail hereinbelow:

In each data simulation, a simulated catheter tip is advanced stepwise, in steps of 1 cm, from the axillary vein/subclavian vein junction through multiple sequential simulated catheter tip positions towards the superior vena cava until the simulated catheter tip reaches the vicinity of the right atrium/superior vena cava junction. The stored tip position location specific corresponding plurality of individual blood pressure waveforms (IBPW) that were obtained at actual multiple sequential catheter tip positions during catheter withdrawal from the right atrium/superior vena cava junction are employed to provide a plurality of simulated IBPW (SIBPW) for the multiple sequential simulated catheter tip positions.

Cross-correlation analysis is performed typically for each of a plurality of heartbeats, typically 16 heartbeats, of each of the simulated sequential catheter tip positions. The cross-correlation analysis is performed between the a. SIBPW and b. the Group Averaged, Multiple Heartbeat Averaged Catheter Tip Position Dependent, Patient Specific Invasive Blood Pressure (GAMHACTPDPSIBP) waveforms. This cross-correlation analysis is performed separately for each tip location in each of the multiple representative waveform groups (MRWG), typically three in number.

The cross correlation analysis indicates which of the Group Averaged, Multiple Heartbeat Averaged Catheter Tip Position Dependent, Patient Specific Invasive Blood Pressure (GAMHACTPDPSIBP) waveforms is closest to the SIBPW at each heartbeat and thus provides a plurality of indications, typically 16 indications, of the Simulated Estimated Catheter Tip Location (SECTL) with respect to the right atrium/superior vena cava junction.

Typically, the median of the plurality of indications of SECTL is calculated. This median provides a Median Simulated Estimated Catheter Tip Location (MSECTL). Such a MSECTL is provided for each of the Multiple Representative Waveform Groups (MRWG). The MSECTLs for each of the Multiple Representative Waveform Groups (MRWG) are calculated for each of the simulated sequential catheter tip positions and stored.

If a plurality of individual blood pressure waveforms (IBPW) for a given simulated catheter tip position were not obtained during catheter withdrawal, a linear interpolation is applied to the MSECTLs.

Preferably the MSECTLs, according to the Group Averaged, Multiple Heartbeat Averaged Catheter Tip Position Dependent, Patient Specific Invasive Blood Pressure (GAMHACTPDPSIBP), for only Group C are employed for the catheter tip location estimation, unless specifically indicated otherwise. As noted above, Group C is the one of the Multiple Representative Waveform Groups (MRWG) in which the ratio of estimated catheter tip location to the measured catheter tip location is sometimes above and sometimes below the identity line 140.

Preferably, when the simulated catheter tip is at a position at which the MSECTL is equal to or less than 2.75 cm from the right atrium/superior vena cava junction, hereinafter referred to as the Simulated ETOLT2.75 position (SETOLT2.75), a procedure for initial definition of the Group Selection Criteria (PIDGSC) is commenced and performed on the data of each of the patients in the representative sample. The Procedure for Initial Definition of the Group Selection Criteria (PIDGSC) preferably includes two stages, PIDGSC-A and PIDGSC-B.

Stage PIDGSC-A involves calculation of the slope of a plot of MSECTL, based on the Group Averaged, Multiple Heartbeat Averaged Catheter Tip Position Dependent, Patient Specific Invasive Blood Pressure (GAMHACTPDPSIBP) for Group C vs. the simulated catheter tip position. Two MSECTLs are plotted, one MSECTL at a simulated catheter tip position equal to SETOLT2.75 and a second MSECTL at a simulated catheter tip position 4 cm further from the heart than SETOLT2.75. This second position is referred to hereinafter as SETOLT2.75+4 cm.

The calculated slopes of the plots of MSECTL, based on the Group Averaged, Multiple Heartbeat Averaged Catheter Tip Position Dependent, Patient Specific Invasive Blood Pressure (GAMHACTPDPSIBP) for Group C vs. the simulated catheter tip position for all patients in the representative sample are divided into preferably three Characteristic Slope Range groups (CSRG), such that each patient in the representative sample group is assigned to one CSRG.

It is noted that as described hereinabove in reference to FIGS. 1C and 1D, each patient in the representative sample group is also assigned to a specific multiple representative waveform group (MRWG) according to a characteristic blood pressure signal propagation rate within the patient's vein having a corresponding blood pressure waveform.

The range boundaries of the three Characteristic Slope Range groups (CSRG) are preferably selected in an iterative manner such that insofar as possible the members in each given MRWG are identical to the members in a corresponding CSRG.

Preferably, three CSRG are defined and are designated as Characteristic Slope Range Group—A (CSRG—A), Characteristic Slope Range Group—B (CSRG—B and Characteristic Slope Range Group—C (CSRG—C). The initial range boundaries for these groups (IRB-CSRG) are designated as IRB-CSRG-A, IRB-CSRG-B & IRB-CSRG-C and are stored for use in subsequent clinical procedures.

The calculated slopes for each of the patients in the representative sample group are associated with one of the multiple representative groups (MRWG) according to the IRB-CSRG to which the patient belongs. The MRWG to which each patient is thus assigned is hereinafter termed an Initial Slope MRWG or ISMRWG.

Stage PIDGSC-B involves calculation of two parameters P1 and P2 for each of the patients in the representative sample group.

P1 is the average of all the stored MSECTLs for a given patient based on the Group Averaged, Multiple Heartbeat Averaged Catheter Tip Position Dependent, Patient Specific Invasive Blood Pressure (GAMHACTPDPSIBP) of Group C.

P2 is the MSECTL for the same patient at the simulated catheter tip position SETOLT2.75+4 cm, based on the Group Averaged, Multiple Heartbeat Averaged Catheter Tip Position Dependent, Patient Specific Invasive Blood Pressure (GAMHACTPDPSIBP) of the ISMRWG.

Plots of P1 vs. P2 are preferably prepared for each Characteristic Slope Range Group (CSRG) and are designated as P1/P2 Plot—A, P1/P2 Plot—B and P1/P2 Plot—C respectively. Examples of these plots appear in FIG. 1F.

Each of the P1/P2 plots is preferably divided into multiple Group Characteristic Domains (GCD) each identified by a capital letter, such as A, B & C. Each of the Group Characteristic Domains (GCD) corresponds to one of the multiple representative waveform groups (MRWG) with which each patient in the representative sample was previously associated.

The Initial Boundaries of the Group Characteristic Domains (GCD) are iteratively defined such that insofar as possible the P1/P2 data of patients in a given MRWG lie within a corresponding at least one GCD. Examples of GCD appear in FIG. 1F.

The Initial Boundaries of each of the Group Characteristic Domains (IBGCD), here designated IBGCD-A, IBGCD-B & IBGCD-C are stored for future use during the clinical procedure.

Simulated P1/P2 data for each of the patients in the representative sample group is associated with one of the multiple representative waveform groups (MRWG) according to the IBGCD to which the patient belongs. This association results in an assignment of the patient to one of the MRWG groups, hereinafter termed the “ISMRWG-B Selected Group”.

The foregoing is a simplified explanation of the Procedure for Initial Definition of the Group Selection Criteria (PIDGSC).

The simulated catheter tip is now further advanced stepwise in steps of 1 cm towards the heart until the simulated catheter tip reaches a position at which the MSECTL corresponding to the ISMRWG-B selected group is equal to or less than 2 cm from the right atrium/superior vena cava junction, hereinafter referred to as the Simulated ETOLT2 position (SETOLT2). A Procedure for Secondary Definition of the Group Selection Criteria (PSDGSC) is carried out based on data stored for each simulated stepwise advancement of the catheter tip. This procedure is similar to the Procedure for Initial Definition of the Group Selection Criteria (PIDGSC) described hereinabove and preferably includes two stages, PSDGSC-A and PSDGSC-B. Secondary range boundaries (SRB-CSRG), corresponding to the IRB-CSRG, are designated as SRB-CSRG-A, SRB-CSRG-B & SRB-CSRG-C and are stored for use in subsequent clinical procedures.

Secondary Boundaries of each of the Group Characteristic Domains (SBGCD), corresponding to the IBGCD here designated SBGCD-A, SBGCD-B & SBGCD-C are stored for future use during the clinical procedure.

The foregoing is a simplified explanation of the establishment of Group Selection Criteria (GSC), which are stored and later used during the clinical procedure.

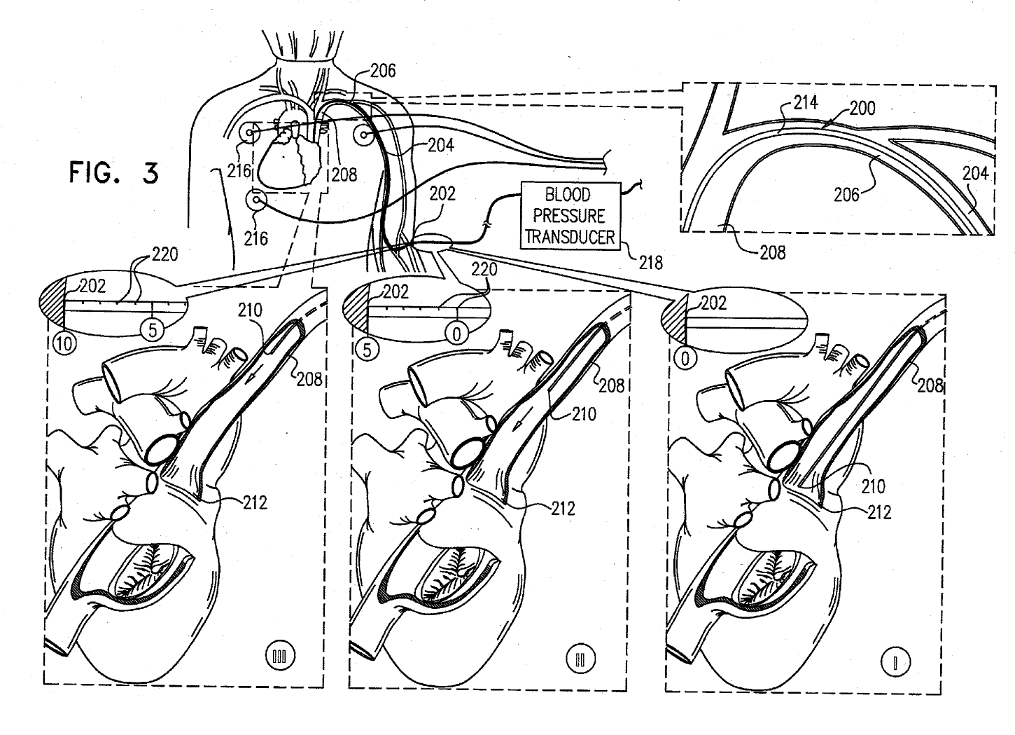

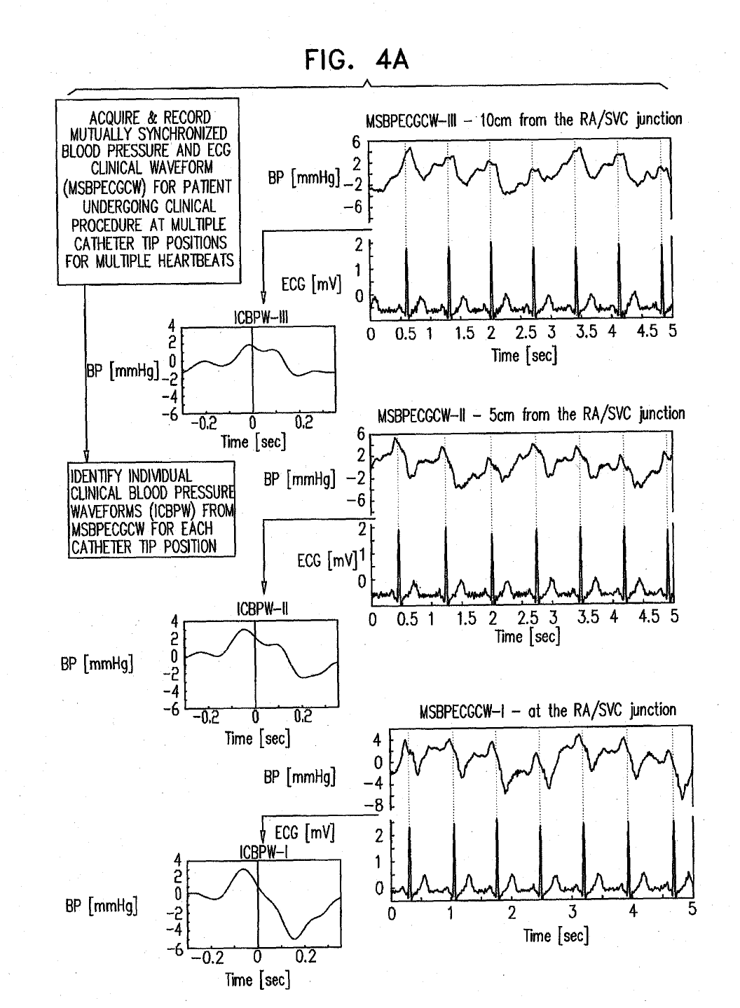

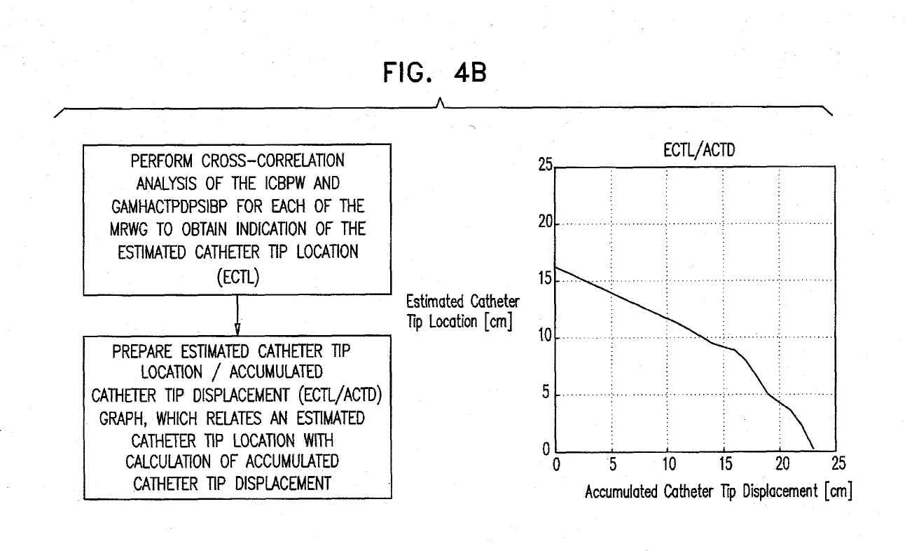

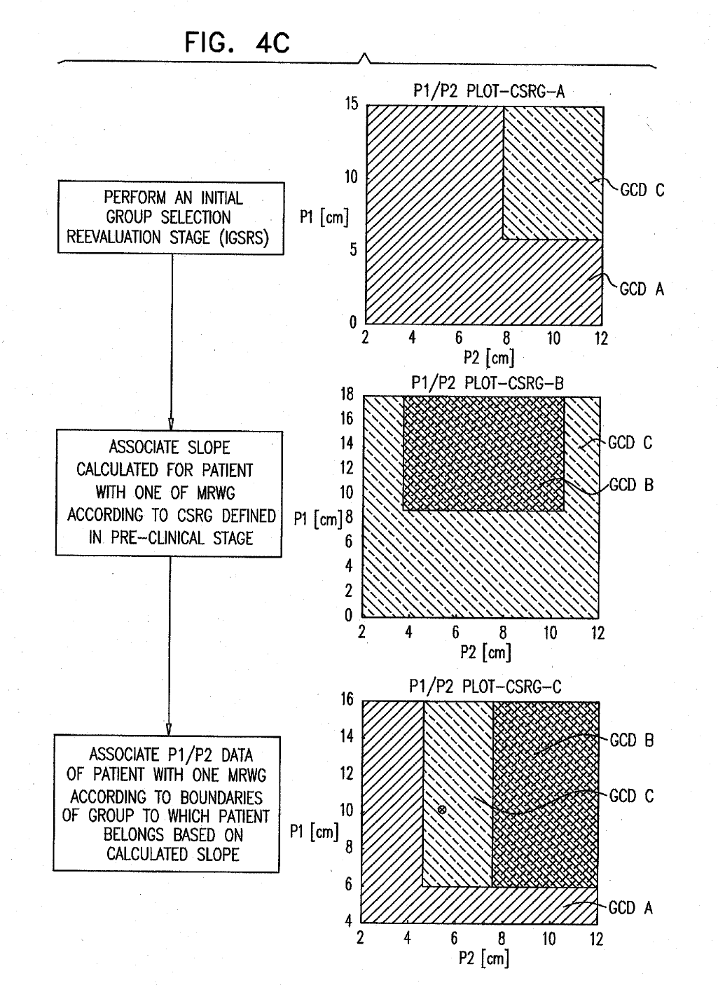

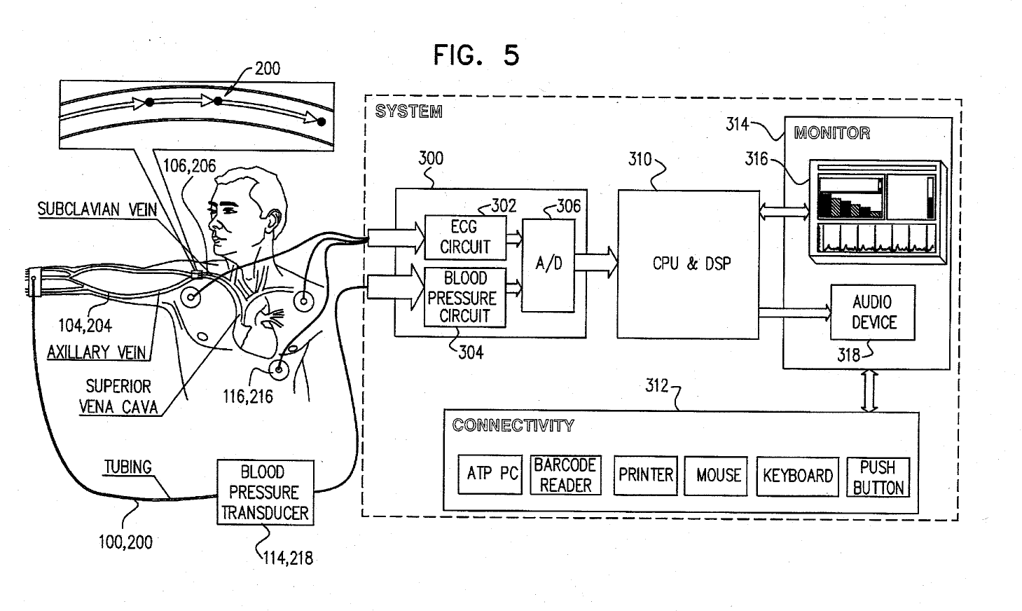

Reference is now made to FIG. 3, which is a simplified illustration of a clinical procedure for precise insertion of a catheter at the right atrium/superior vena cava junction, and to FIGS. 4A-4C, which together are a simplified illustrated flowchart illustrating a methodology for precise insertion of a catheter at the right atrium/superior vena cava junction in accordance with a preferred embodiment of the present invention. The following discussion is a simplified explanation of the functionality of a preferred embodiment of the present invention in a clinical environment. This discussion demonstrates how an embodiment of the present invention employs the stored data which was obtained as described hereinabove.

As seen in FIG. 3, employing the system and methodology of the present invention, an operator, such as a nurse, inserts a catheter 200 in a conventional manner, at an insertion location 202 at a patient's arm and through a vein in the arm to the axillary vein 204, thence to the subclavian vein 206 into the superior vena cava 208 until the tip 210 of the catheter reaches the right atrium/superior vena cava junction 212. During this procedure the parameters of the patient's heartbeat in the form of an ECG signal are received and recorded as are the parameters of the patient's blood pressure.

Use of the system and methodology of the present invention may obviate verification of the actual position of the tip 210 at the right atrium/superior vena cava junction 212 by X-ray imaging.

Beginning typically when the catheter tip 210 is at the junction of the axillary vein and the subclavian vein, indicated by reference numeral 214, and thereafter, as the catheter tip 210 is stepwise displaced towards the right atrium/superior vena cava junction 212 through multiple sequential catheter tip positions, the blood pressure waveform and the ECG waveform of the patient are received in mutual synchronization, where preferably the R wave peak of the ECG waveform represents a fiducial point in time with respect to which the blood pressure waveform is measured.

As seen in FIG. 3, the catheter 200 typically has markings, generally designated by reference numeral 220, at 1 cm intervals therealong, which markings can be readily seen by the operator. Enlargement III in FIG. 3 shows a typical point during the insertion procedure, at which point the catheter marking 10 cm appears at the insertion location 202. It is appreciated that when the catheter tip is at the junction of the axillary vein and the subclavian vein 214, a catheter marking greater than 10 cm, for example, between 15-20 cm, is seen at the insertion location 202.

Typically, the ECG waveform is acquired by several surface electrodes 216 that are positioned on the patient's skin, for example on the patient's chest, as seen in FIG. 3, or, alternatively, on the patient's right arm, left arm and left leg. Typically, the blood pressure waveform at the catheter tip 210 is acquired by a blood pressure transducer 218 at a proximal end of the catheter 200, the blood pressure waves passing through liquid in the catheter 200 from the tip 210 to the transducer 218. An example of Mutually Synchronized Blood Pressure and ECG Clinical Waveforms (MSBPECGCW) for a typical patient appears at MSBPECGCW-III in FIG. 4A. Typically, the mutually synchronized blood pressure waveform and ECG are recorded for a plurality of heartbeats, typically at least 16 heartbeats, at each sequential position of the catheter tip 210.

Three non-sequential typical positions of the catheter tip 210 are illustrated in FIG. 3, at enlargements III, II and I respectively, and typical Mutually Synchronized Blood Pressure and ECG Clinical Waveforms (MSBPECGCW) for these three positions appear respectively at MSBPECGCW-III, MSBPECGCW-II and MSBPECGCW-I in FIG. 4A.

It is appreciated that the non-sequential typical positions of the catheter tip 210 illustrated in FIG. 3, at enlargements III, II and I, respectively, approximately correspond to the non-sequential typical positions of the catheter tip 110 illustrated in FIG. 2 at enlargements III, II and I, respectively.

For each Mutually Synchronized Blood Pressure and ECG Clinical Waveforms (MSBPECGCW) acquired and recorded for the patient at each catheter tip position, a plurality of Individual Clinical Blood Pressure Waveforms (ICBPW) is preferably generated by dividing the blood pressure waveform recorded for the plurality of heartbeats into individual blood pressure waveforms of single heartbeat length, using the R wave peak of the ECG waveform as a fiducial point in time with respect to the blood pressure waveform. The individual clinical blood pressure waveforms (ICBPW) are bounded in time by a fixed time window, typically of duration approximately 660 ms, which preferably opens 300 ms prior to each detected R wave peak in the Mutually Synchronized Blood Pressure and ECG Clinical Waveforms (MSBPECGCW). Additionally, the individual blood pressure waveforms (IBPW) are preferably filtered using conventional filtering techniques, such as Finite Impulse Response (FIR). Typical individual clinical blood pressure waveforms (ICBPW), corresponding to MSBPECGCW-I, MSBPECGCW-II and MSBPECGCW-III, are shown as ICBPW-I, ICBPW-II and ICBPW-III in FIG. 4A.

Cross-correlation analysis is performed, typically in response to an operator actuation, at each of the sequential catheter tip positions. In response to the operator actuation, the system performs cross-correlation of the a. ICBPW and b. the Group Averaged, Multiple Heartbeat Averaged Catheter Tip Position Dependent, Patient Specific Invasive Blood Pressure (GAMHACTPDPSIBP) waveforms. This cross-correlation analysis is performed separately for each of the multiple representative waveform groups, typically three in number.

The cross correlation analysis takes place during time intervals between successive heartbeats for a plurality of heartbeats, typically a total of 16 heartbeats.

The cross correlation analysis indicates which of the Group Averaged, Multiple Heartbeat Averaged Catheter Tip Position Dependent, Patient Specific Invasive Blood Pressure (GAMHACTPDPSIBP) waveforms is closest to the CBPW at each heartbeat and thus provides a plurality of indications, typically 16 indications, of the Estimated Catheter Tip Location (ECTL) with respect to the right atrium/superior vena cava junction.

An Estimated Catheter Tip Location/Accumulated Catheter Tip Displacement (ECTL/ACTD) graph is prepared which relates the estimated location of the catheter tip 210 as derived from the synchronized Multiple Heartbeat Averaged, Catheter Tip Position Dependent, Patient Specific Invasive Blood Pressure (MHACTPDPSIBP) and ECG waveforms with the calculation of the accumulated catheter tip 210 displacement, based on the steps performed by the operator. An example of an Estimated Catheter Tip Location/Accumulated Catheter Tip Displacement graph for the patient appears in FIG. 4B and is designated ECTL/ACTD.

Typically, the median of the above plurality of indications of ECTL is calculated. This median provides a Median Estimated Catheter Tip Location (MECTL). Such a MECTL is provided for each of the Multiple Representative Waveform Groups (MRWG). The MECTLs for each of the Multiple Representative Waveform Groups (MRWG) are stored for each of the sequential catheter tip positions.

Preferably the MECTLs calculated according to the Group Averaged, Multiple Heartbeat Averaged Catheter Tip Position Dependent, Patient Specific Invasive Blood Pressure (GAMHACTPDPSIBP) for only Group C are employed for catheter tip location estimation, unless specifically indicated otherwise. As noted above, Group C is the one of the Multiple Representative Waveform Groups (MRWG) in which the ratio of estimated catheter tip location to the measured catheter tip location is sometimes above and sometimes below the identity line.

In the clinical procedure, starting from when the catheter tip 210 is located at the axillary vein/subclavian vein junction 214, the system, based on the catheter tip location estimation which is obtained as described hereinabove, instructs the operator to sequentially advance the catheter tip 210 in steps of decreasing length. Normally the steps start at 4 cm and decrease to 2 cm when the MECTL is less than 10 cm from the right atrium/superior vena cava junction and decrease to 1 cm when the MECTL is less than 5 cm from the right atrium/superior vena cava junction.

Preferably, when the catheter tip 210 is at a position at which the MECTL is calculated as being equal to or less than 2.75 cm from the right atrium/superior vena cava junction, hereinafter referred to as the ETOLT2.75 position, an initial group selection reevaluation stage (IGSRS) is commenced. The initial group selection reevaluation stage (IGSRS) preferably includes two sub-stages, IGSRS-A and IGSRS-B:

IGSRS-A involves calculation of the slope of a plot of two stored or currently obtained MECTLs of the patient, based on the Group Averaged, Multiple Heartbeat Averaged Catheter Tip Position Dependent, Patient Specific Invasive Blood Pressure (GAMHACTPDPSIBP) for Group C, at the ETOLT2.75 position and at a catheter tip position 4 cm further from the heart than ETOLT2.75. This second position is referred to hereinafter as ETOLT2.75-4 cm.

This calculated slope for the patient is associated with one of the multiple representative groups (MRWG) according to the IRB-CSRG to which the patient belongs, resulting in an initial slope MRWG (ISMRWG).

Sub-Stage IGSRS-B involves calculation of two parameters P1 and P2.

P1 is the average of all the stored MECTLs for the patient based on the Group Averaged, Multiple Heartbeat Averaged Catheter Tip Position Dependent, Patient Specific Invasive Blood Pressure (GAMHACTPDPSIBP) of Group C. Preferably this average is calculated using an interpolation of the stored MECTLs which provides MECTL values for steps, of equal length, from the axillary vein/subclavian vein junction 214 towards the heart.

P2 is the MECTL for the patient at the catheter tip position ETOLT2.75-4 cm, based on the Group Averaged, Multiple Heartbeat Averaged Catheter Tip Position Dependent, Patient Specific Invasive Blood Pressure (GAMHACTPDPSIBP) of the ISMRWG.

P1/P2 data of the patient is associated with one of the multiple representative groups (MRWG) according to the IBGCD to which the patient belongs. This association results in an assignment of the patient to one of the MRWG groups hereinafter termed the “IMRWG-B Selected Group”.

The foregoing is a simplified explanation of the initial group selection reevaluation stage (IGSRS).

The catheter tip 210 is now advanced stepwise towards the heart in steps of 1 cm until the catheter tip 210 reaches a position at which the MECTL corresponding to the IMRWG-B selected group is equal to or less than 2 cm, hereinafter referred to as the ETOLT2 position. At this point, a Secondary Group Selection Reevaluation Stage (SGSRS) is carried out based on the patient's stored or currently obtained MECTLs. The SGSRS is similar to the Initial Group Selection Reevaluation Stage (IGSRS) described hereinabove and preferably includes two sub-stages, SGSRS-A and SGSRS-B:

SGSRS-A involves calculation of the slope of a plot of two stored or currently obtained MECTLs of the patient, based on the Group Averaged, Multiple Heartbeat Averaged Catheter Tip Position Dependent, Patient Specific Invasive Blood Pressure (GAMHACTPDPSIBP) for Group C, at the ETOLT2 position and at a catheter tip position 4 cm further from the heart than ETOLT2. This second position is referred to hereinafter as ETOLT2-4 cm.

This calculated slope for the patient is associated with one of the multiple representative groups (MRWG) according to the IRB-CSRG to which the patient belongs, resulting in an initial slope MRWG (ISMRWG).

Sub-Stage IGSRS-B involves calculation of two parameters P1 and P2.

P1 is the average of all the stored MECTLs for the patient based on the Group Averaged, Multiple Heartbeat Averaged Catheter Tip Position Dependent, Patient Specific Invasive Blood Pressure (GAMHACTPDPSIBP) of Group C. Preferably this average is calculated using an interpolation of the stored MECTLs which provides MECTL values for steps, of equal length, from the axillary vein/subclavian vein junction 214 towards the heart.

P2 is the MECTL for the patient at the catheter tip position ETOLT2-4 cm, based on the Group Averaged, Multiple Heartbeat Averaged Catheter Tip Position Dependent, Patient Specific Invasive Blood Pressure (GAMHACTPDPSIBP) of the ISMRWG.

P1/P2 data of the patient is associated with one of the multiple representative groups (MRWG) according to the IBGCD to which the patient belongs. This association results in an assignment of the patient to one of the MRWG groups hereinafter termed the “SMRWG-B Selected Group”. This association is shown in FIG. 4C, which shows the P1/P2 Plots and associated GCDs defined in the pre-clinical stage as described hereinabove with reference to FIG. 1F overlaid with the GCD selected for the patient based on the slope value and P1/P2 value for the patient. In the illustrated example the “SMRWG-B Selected Group” would correspond to MRWG-C.

At this stage the stored calculated MECTLs of the patient for the SMRWG-B Selected Group at 1 cm intervals typically between ETOLT2-7 cm and ETOL2-2 cm are searched to find a calculated catheter tip location at which the stored MECTL is closest to but does not exceed 2.

If such a calculated catheter tip location is found the desired target location is identified as being one centimeter closer to the heart than the above calculated catheter tip location at which the stored MECTL is closest to but does not exceed 2.

If such a calculated catheter tip location is not found typically between ETOLT2-7 cm and a catheter tip location 5 cm closer towards the heart, the desired target location is identified as being one centimeter closer to the heart than ETOLT2-2 cm.

The catheter tip 210 is then displaced to the calculated desired target location and the operator is informed that the catheter tip 210 is located at the target.

Reference is now made to FIG. 5, which is a simplified block diagram illustration of the system of a preferred embodiment of the present invention. In addition to the system components described hereinabove with reference to FIGS. 2 and 3, which are identified by the same reference numerals, the system includes input signal amplification and digitization circuitry 300 which receives inputs from electrodes 116/216 and from blood pressure transducer 114/218. The inputs from electrodes 116/216 are provided to ECG signal filtering and amplification circuitry 302 and the inputs from blood pressure transducer 114/218 are supplied to blood pressure filtering and amplification circuitry 304. Circuitry 302 and circuitry 304 both output to A/D circuitry 306, which provides a digital bit stream output to computation circuitry 310.

Computation circuitry 310 typically comprises a CPU chip, a DSP and memory for storing the computed values described hereinabove and is operative to perform the various computations described hereinabove with reference to FIGS. 1A-4C. Computation circuitry 310 preferably interfaces with a user input interface 312 and a user audio-visual interface 314, preferably including a display 316 and an audio output device 318, such as a speaker.

The system preferably provides one or more instructions to an operator, directing the operator to displace the catheter a suitable distance until a desired catheter tip location is reached, based on the output of the computations described hereinabove which provide an indication of the current catheter tip location. The operator instructions are typically provided via at least one of display 316 and audio output device 318.

It will be appreciated by persons skilled in the art that the present invention is not limited by what has been particularly shown and described hereinabove. Rather the present invention includes both combinations and subcombinations of various features described herein and improvements and variations which would occur to persons skilled in the art upon reading the foregoing description and which are not in the prior art.

1. A system for providing indications of location of a catheter tip within a patient, the system comprising:
a memory storing synthesized blood pressure waveform parameters representative of a first plurality of catheter tip locations in a second plurality of representative patients;
real time patient blood pressure waveform parameter acquisition circuitry operative to acquire real time blood pressure waveform parameters of a patient currently undergoing catheterization, which parameters contain information useful in ascertaining the location of a catheter tip in the patient; and
real time patient catheter tip location ascertaining circuitry operative to receive said real time patient blood pressure waveform parameters from said acquisition circuitry and to employ said synthesized blood pressure waveform parameters for providing a real time indication of catheter tip location in the patient.

2. A system for providing indications of location of a catheter tip within a patient according to claim 1 and wherein said synthesized blood pressure waveform parameters are based on waveform parameters from multiple ones of said plurality of representative patients.

3. A system for providing indications of location of a catheter tip within a patient according to claim 1 and wherein said real time patient blood pressure waveform parameter acquisition circuitry is operative to acquire real time blood pressure waveform parameters of said plurality of representative patients and said system also comprises:
patient blood pressure waveform parameter grouping circuitry operative to group said plurality of representative patients into multiple groups; and
blood pressure waveform parameter synthesizing circuitry operative to generate said synthesized blood pressure waveform parameters for each of said multiple groups based on said blood pressure waveform parameters of said plurality of representative patients.

4. A system for providing indications of location of a catheter tip within a patient according to claim 1 and wherein:
said synthesized blood pressure waveform parameters are grouped into multiple groups having mutually differing relationships between measured and estimated catheter tip locations; and
said real time patient catheter tip location ascertaining circuitry is operative to associate parameters of a patient with parameters of one of said multiple groups in order to enhance accuracy of indication of catheter tip location based on said real time patient blood pressure waveform parameters.

5. A system for providing indications of location of a catheter tip within a patient according to claim 4 and wherein said multiple groups differ in characteristic blood pressure signal propagation rate within a vein of the patient.

6. A system for providing indications of location of a catheter tip within a patient according to claim 1 and wherein said real time patient catheter tip location ascertaining circuitry is operative to match said real time patient blood pressure waveform parameters with at least one of said synthesized blood pressure waveform parameters for providing a real time indication of catheter tip location in the patient.

7. A system for providing indications of location of a catheter tip within a patient according to claim 1 and also comprising electrodes located on the patient skin, said electrodes being operative to provide ECG waveform parameters of said patient.

8. A system for providing indications of location of a catheter tip within a patient according to claim 7 and wherein:
said real time blood pressure waveform parameters are recorded in synchronization with said ECG waveform parameters; and
an R wave peak of said ECG waveform parameters represents a fiducial point in time with respect to which said real time blood pressure waveform parameters are measured.

9. A system for providing indications of location of a catheter tip within a patient according to claim 8 and wherein a time shift between said real time blood pressure waveform parameters and said R wave peak of said ECG waveform parameters provides a real time indication of catheter tip location in the patient.

10. A system for providing indications of location of a catheter tip within a patient according to claim 1 and wherein said real time patient blood pressure waveform parameter acquisition circuitry comprises a pressure transducer operatively coupled to said catheter tip.

11. A system for providing indications of location of a catheter tip within a patient according to claim 1 and wherein said real time patient catheter tip location ascertaining circuitry employs cross-correlation analysis.

12. A system for providing indications of location of a catheter tip within a patient according to claim 1 and wherein said real time patient catheter tip location ascertaining circuitry is also operative to provide at least one catheter tip insertion instruction to an operator based on said real time indication of catheter tip location in the patient.

13. A system for providing indications of location of a catheter tip within a patient according to claim 12 and also comprising at least one of a display and an audio output device and wherein said at least one catheter tip insertion instruction is provided to said operator via said at least one of a display and an audio output device.

14. A method for providing indications of location of a catheter tip within a patient, the method comprising:
storing synthesized blood pressure waveform parameters representative of a first plurality of catheter tip locations in a second plurality of representative patients;
acquiring real time patient blood pressure waveform parameters of a patient currently undergoing catheterization, which parameters contain information useful in ascertaining the location of a catheter tip in the patient; and
ascertaining a real time indication of catheter tip location in the patient by employing said real time patient blood pressure waveform parameters and said synthesized blood pressure waveform parameters.

15. A method for providing indications of location of a catheter tip within a patient according to claim 14 and also comprising automatically generating said synthesized blood pressure waveform parameters based on waveform parameters from multiple ones of said plurality of representative patients.

16. A method for providing indications of location of a catheter tip within a patient according to claim 15 and wherein said automatically generating comprises:
acquiring real time patient blood pressure waveform parameters of said plurality of representative patients;
grouping said plurality of representative patients into multiple groups; and
generating said synthesized blood pressure waveform parameters based on said blood pressure waveform parameters for each of said multiple groups.

17-19. (canceled)

20. A method for providing indications of location of a catheter tip within a patient according to claim 14 and also comprising acquiring real time patient ECG waveform parameters of said patient.

21. A method for providing indications of location of a catheter tip within a patient according to claim 20 and wherein:
said acquiring real time patient blood pressure waveform parameters of a patient and said acquiring real time patient ECG waveform parameters of said patient comprises synchronized acquiring; and
an R wave peak of said ECG waveform parameters represents a fiducial point in time with respect to which said real time blood pressure waveform parameters are measured.

22. A method for providing indications of location of a catheter tip within a patient according to claim 21 and wherein said ascertaining comprises ascertaining a time shift between said real time blood pressure waveform parameters and said R wave peak of said ECG waveform parameters.

23. A method for providing indications of location of a catheter tip within a patient according to claim 14 and wherein said ascertaining a real time indication of catheter tip location in the patient comprises employing a cross-correlation analysis.

24-25. (canceled)
